(19) United States
(12) Patent Application Publication
(10) Pub. No.: US 2007/0248950 A1
(43) Pub. Date: Oct. 25, 2007

(54) Supported polydiacetylene 3-D arrays for flourescent or phosphorescent detection

(75) Inventors: Mary A. Reppy, Wilmington, DE (US); Bradford A. Pindzola, Wilmington, DE (US); Bethel Sharma, Wilmington, DE (US); Marianne Zecher, Wilmington, DE (US)

Correspondence Address: Connolly Bove lodge & Hutz LLP, Suite 800, 1990 M Street, N.W., Washington, DC, 20036, US

(73) Assignee: Analytical Biological Services, Inc., Wilmington, DE (US)

(21) Appl. No.: 11/406,525

(22) Filed: Apr. 19, 2006

(51) Int. Cl. C12Q 1/70; C12Q 1/68; G01N 33/53; C12M 3/00

(52) U.S. Cl. 435/5; 435/6; 435/7.1; 435/287.2

(57) ABSTRACT

Supported three-dimensional arrays of a polydiacetylene backbone bonded to and supported by a solid support in a sample are provided. The bonding is achieved by providing a support having thiol and/or amine groups and epoxy groups and/or maleimide groups, and a polydiacetylene backbone containing the other of thiol and/or amine groups or the epoxy and/or maleimide groups for reacting with the corresponding thiol groups or epoxy groups and/or maleimide groups on the support; provided that when an amine is used the other group is an epoxy; and/or having a layer of a polyelectrolyte on the support; and/or having a multiple atom linker between the three-dimensional array of the polydiacetylene backbone and the solid support. The supported three-dimensional arrays can be used for detecting an analyte; for evaluating the organic/water partition coefficient and/or oral absorptivity and/or ability of a compound to diffuse into cell membranes and/or transcellular permeability properties of a compound and/or the ionization state of a compound and/or the volume of distribution of a compound and/or the distribution of a compound into different tissues and/or the partitioning of a compound into cell organelles by monitoring the change in the fluorescence or phosphorescence upon exposure to the compound and optionally comparing it to a known change in fluorescence or phosphorescence, respectively. The supported three-dimensional arrays can also be used to evaluate the ability of a compound to bind to a protein and for the detection of a plurality of different species.

### FEDERALLY SPONSORED RESEARCH OR DEVELOPMENT

Inventions in this disclosure were partially supported by a Phase II SBIR grant from the National Science Foundation (DMI-0239587) and a Phase II STTR grant from the Army Research Office (W911NF-O4-C-0132).

### TECHNICAL FIELD

The present disclosure relates to supported three-dimensional arrays of a polydiacetylene backbone bonded to and supported on a solid support. Supported three-dimensional arrays of this disclosure are capable of achieving a tenacious bond to the support without destroying the ability of the three-dimensional arrays to be used in detection methods that rely upon or fluorescence and/or phosphorescence

Supported three-dimensional arrays of this disclosure can be used for detecting of an analyte in a sample by measuring a change in fluorescence and/or phosphorescence. Also, supported three-dimensional arrays of this disclosure can be used for evaluating at least one of the ionization state of a compound, the organic/water partition coefficient and lipophilicity, oral absorption, the volume of distribution of a compound, the distribution of a compound into different tissues, the ability of a compound to diffuse into cell membranes and the partitioning of a compound into cell organelles, by measuring the effect on the array from exposure to the compound by detecting the change in fluorescence or phosphorescence of the array. Supported three-dimensional arrays of this disclosure can be used for evaluating the binding of a compound to a protein. Supported three-dimensional arrays of this disclosure can be used for detecting a plurality of different species.

### BACKGROUND

Three-dimensional arrays of polydiacetylene backbones, such as polydiacetylene liposomes and other colloids, have been proposed for use as materials for calorimetric and fluorescence detection of analytes. Previously these arrays were generally prepared and used in solution. Polydiacetylene liposomes entrapped in sol gels have also been proposed for colorimetric sensors (Yamanaka S. et al, Langmuir, 1997, 13, 5049-5053; Gill I. and Ballesteros A., Angew. Chem. Int. Ed. 2003, 42, 3254-3267, Charych et al, U.S. Pat. Nos. 6,485,987 and 6,022,748, “Sol-Gel Matrices for Direct Colorimetric Detection of Analytes”, both 2/8/200). However, transport of analytes through the gels is difficult and slow and has limited the utility of these materials. Polydiacetylene liposomes linked to glass slide surfaces have been more recently described (Kim J-M et al, Adv. Mater. 2003, 15(13), 118-1121; Shim H. Y. et al, Mater. Sci. Eng. C, 2004, 24, 157-161; Kim J-M et al., J. Am. Chem. Soc. 2005, 127, 17580-17581), but the methods of attachment leave room for improvement.

Polydiacetylene arrays are disclosed in PCT/US01/008790; U.S. Pat. No. 6,984,528; US Patent Application Publication US2004-0023303; and PCT/US05/28082; all of which are assigned to Analytical Biological Services, Inc. and incorporated herein by reference that are useful for various analytical or detection techniques that rely upon fluorescence and/or phosphorescence.

### SUMMARY OF DISCLOSURE

The present disclosure relates to providing supported three-dimensional array of a polydiacetylene backbone bonded to and supported by a solid support by certain techniques that are capable of achieving a tenacious bond without destroying the ability of the three-dimensional array to be used in detection methods that rely upon or fluorescence and/or phosphorescence.

According to one aspect of the present disclosure, a supported three-dimensional array of a polydiacetylene backbone bonded to and supported by a solid support is provided. The support has at least one member selected from the group consisting of thiol group and amine group or at least one member selected from the group consisting of epoxy groups and maleimide groups on the surface that is to be in contact with the three-dimensional array. The polydiacetylene backbone array contains the other of thiol group and/or amine group or the epoxy and/or maleimide groups for reacting with the corresponding thiol group and/or amine group or epoxy groups and/or maleimide groups on the solid support; provided that when an amine is used the other group is an epoxy. In other words, the reactive combinations are: thiol and epoxy; thiol and maleimide; amine and epoxy.

According to another aspect of the present disclosure a supported three-dimensional array of a polydiacetylene backbone bonded to and supported by a solid support is provided. The solid support has a layer of polyelectrolyte on the surface that is to be in contact with the three-dimensional array. According to this aspect of the present disclosure, the solid support is not restricted to a support having the thiol and/or amine groups or epoxy groups and/or maleimide groups thereon.

A still further aspect of this disclosure is concerned with a supported three-dimensional array of a polydiacetylene backbone bonded to and supported by a solid support. A multiple atom linker is located between the three-dimensional array of the polydiacetylene backbone and the solid support. According to this aspect of the present disclosure, the solid support is not restricted to the support having the thiol and/or amine groups or epoxy groups and/or maleimide groups thereon.

Supported three-dimensional arrays disclosed above can be used in the methods disclosed in PCT/US01/008790; U.S. Pat. No. 6,984,528; US Patent Application Publication US2004-0023303; and PCT/US05/28082; all of which are incorporated herein by reference.

Another aspect of the present disclosure is a method for the detection of an analyte in a sample, which comprises contacting the sample to be tested with any of the above disclosed supported three-dimensional arrays;
and detecting the change in fluorescence or phosphorescence to indicate the presence of the analyte. When to be employed for this purpose, the array has incorporated therein a substrate that has direct affinity for an analyte or can function as a binder to an analyte or can react with an analyte.

A further aspect of the present disclosure relates to a method for evaluating the binding of a compound to a protein, which comprises exposing any of the above disclosed supported three-dimensional arrays to a solution of the compound to be evaluated post or during exposure to the protein;

measuring the effect on the array of the compound to be evaluated by detecting the change in fluorescence or phosphorescence,

comparing the change to changes in fluorescence or phosphorescence from exposure of the array of the kind being used to a reference compound in solution.

Another aspect of the present disclosure is concerned with a method for detecting a plurality of different species. The present disclosure provides a method for screening a plurality of samples containing different species, which comprises exposing an attached three-dimensional array of a polydiacetylene backbone to the samples to be evaluated; wherein the array is capable of hetero-detection:

detecting the change in fluorescence or phosphorescence of the array, and

comparing the change to a previously determined change in fluorescence or phosphorescence of the array to determine whether the species are present in the samples. In a further refinement, comparison with calibration curves allows determination of the concentration of the species.

Another aspect of the present disclosure is concerned with a method for evaluating at least one of the ionization state of a compound, the organic/water partition coefficient and lipophilicity, oral absorption, the volume of distribution of a compound, the distribution of a compound into different tissues, the ability of a compound to diffuse into cell membranes and the partitioning of a compound into cell organelles, which comprises exposing any of the above disclosed supported three-dimensional arrays to the compound to be evaluated; and

measuring the effect on the array by detecting the change in fluorescence or phosphorescence of the array.

Still other objects and advantages of the present disclosure will become readily apparent by those skilled in the art from the following detailed description, wherein it is shown and described only the preferred embodiments, simply by way of illustration of the best mode. As will be realized, the disclosure is capable of other and different embodiments, and its several details are capable of modifications in various obvious respects, without departing from the disclosure. Accordingly, the description is to be regarded as illustrative in nature and not as restrictive.

### BRIEF DESCRIPTION OF DRAWINGS

FIG. 1 illustrates some sequences suitable for preparing the supported and attached 3-D arrays of the present disclosure.

FIG. 2 shows the rise in emission at 640 nm of supported liposomes exposed to PLA₂ alone (red 10 μM, pink 2.5 μM and light pink 0.1 μM), and to 10 μM PLA₂ with 12.7 μM scalaradial (green). The buffer control is shown in yellow, and the scalaradial alone in light blue.

FIG. 3 shows an assay of E. coli with polymerized colloids attached to a treated filter plate.

FIG. 4 shows a Ricin assay with polymerized colloids attached to a treated filter plate.

### DESCRIPTION OF PREFERRED AND VARIOUS EMBODIMENTS

According to one aspect of the present disclosure, a supported three-dimensional array of a polydiacetylene backbone bonded to and supported by a solid support is provided. The solid support has at least one member selected from the group consisting of thiol group and amine group or at least one member selected from the group consisting of epoxy groups and maleimide groups, and preferably epoxy groups on the surface that is to be in contact with the three-dimensional array. The solid support is preferably a glass support. The glass surfaces may be slides, waveguides and/or other extended planar surfaces, or glass fibers, or membranes made of glass fibers or from porous glass. The glass surfaces may be colorless or colored. The glass surfaces may be black. The glass surfaces may be transparent or opaque or semi-transparent. Preferably, the glass surface is porous and most preferably a porous glass membrane. Typically porous glass membranes have thicknesses of about 200 g to about 1 mm.

The polydiacetylene backbone contains the other of thiol and/or amine groups or the epoxy and/or maleimide groups for reacting with the corresponding thiol and/or amine groups or epoxy groups and/or maleimide groups on the solid support; provided that when an amine is used the other group is an epoxy. In the preferred aspects the thiol groups are present in the array.

The array optionally has incorporated therein a substrate that has direct affinity for an analyte or can function as a binder to an analyte or can react with an analyte, depending upon the intended use of the array. The bonding can be covalent or non-covalent.

Diacetylene colloidal (i.e., 3-D arrays of diacetylene backbones) solutions are prepared with the diacetylene colloids containing a thiol group or component that can react with the functional epoxide groups on the glass. Colloids include, but are not limited to, micelles, liposomes, vesicles, tubules, ribbons, nano-tubes etc, and aggregates of these. Colloids may or may not be polymerized before attachment to the glass surfaces. The diacetylene colloids may be photopolymerized to create 3-D arrays of polydiacetylene backbones at any stage of the procedure, including before attachment to the surface.

Examples of compounds incorporated in the three-dimensional array containing a thiol group that are suitable for this disclosure are 1,2-dipalmitoyl-sn-glycerophosphatidylethanethiol, 1-dodecanethiol, 1-undecanethiol, 1-decane thiol, diacetylenes with terminal thiol groups and any other thiol compound that has a hydrophobic tail that can incorporate into the hydrophobic regions of the diacetylene and polydiacetylene arrays. The tail, may, or may not be polymerizable.

Examples of compounds incorporated in the three-dimensional array containing a amine group that are suitable for this disclosure are 1-amino-10,12-pentacosadiyne, 1,2-dipalmitoyl-sn-glycerophosphatidylethanolamine, 1,2-distearoyl-sn-glycerophosphatidylethanolamine, 1,2-dimyristoyl-sn-glycerophosphatidylethanolamine, 1,2-oleoyl-sn-glycerophosphatidylethanolamine, 1-dodecaneamine, 1-undecaneamine, 1-decaneamine, diacetylenes with terminal amine groups, and any other amine compound that has a hydrophobic tail that can incorporate into the hydrophobic regions of the diacetylene and polydiacetylene arrays. The tail, may, or may not be polymerizable.

Examples of compounds containing epoxy groups suitable for this disclosure and are 3-glycidoxy propyl trimethoxy silane, 3-glycidoxypropyl triethoxy silane, 3-glycidoxypropylmethyl dimethoxy silane, 3-glycidoxypropylmethyl diethoxy silane, epoxycyclohexylethyltrimethoxysilane, other mono, di and trialkoxy silanes (typically 1-3 carbon atom alkoxys), and mono, di and trihalo silanes (including mono and di chloro silanes), that incorporate epoxy groups.

Examples of compounds incorporated in the three-dimensional array containing a maleimide group that are suitable for this disclosure include: N-(4-(p-maleimidophenyl)butyryl)-1,2-dipalmitoyl-sn-glycero-3-phosphoethanolamine, N-(4-(p-maleimidophenyl)butyryl)-1,2-distearoyl-sn-glycero-3-phosphoethanolamine N-(3-maleimidobenzoyl)-1,2-dipalmitoyl-sn-glycero-3-phosphoethanolamine, N-(4-(p-maleimidophenyl)butyryl)-1,2-distearoyl-sn-glycero-3-phosphoethanolamine, N-(6-maleimidohexanoyl)-1,2-distearoyl-sn-glycero-3-phosphoethanolamine, 11-[4-(p-maleimidophenyl)butyryl]amino-3,6,9-trioxaundecyl 1,2-distearoyl-sn-glycero-3-phosphate, 1-{8-[4-(p-maleimidophenyl)butyryl]amino-3,6-dioxaoctyl}-2,3-distearyl glyceryl-di-ether, N-{ω-[4-(p-maleimidophenyl)butyrylamino]poly(ethylene glycol) 2000}-1,2-distearoyl-snglycero-3-phosphoethanolamine, and N-{ω-[4-(p-maleimidophenyl)butyrylamino]poly(ethylene glycol) 5000}-1,2-distearoyl-snglycero-3-phosphoethanolamine, diacetylenes with terminal maleimide groups, and any other maleimide compound with a hydrophobic tail that can incorporate in the hydrophobic regions of the diacetylene and polydiacetylene arrays. The tail, may, or may not be polymerizable.

Examples of compounds incorporated in the three-dimensional array containing an epoxy group include 1,2-epoxy-tetradecane, 1,2-epoxy-dodecane, 1,2-epoxy-decane, diacetylenes with 1,2-epoxy terminal groups and any other epoxy compound with a hydrophobic tail that can incorporate in the hydrophobic regions of the diacetylene and polydiacetylene arrays.

Examples of thiol compounds that can be coated on the solid support are mercaptoalkyldi- and tri-alkoxy silanes such as mercaptopropyltrimethoxy silane, mercaptopropyltriethoxy silane and mercaptopropylmethyldimethoxy silane. Examples of amine compounds that can be coated on the solid support are aminoalkyldi-and-trialkyoxy silanes such as 3-aminopropyltrimethoxysilane and 3-aminopropyltriethoxysilane. The thiols and/or amines or epoxy and/or maleimide when incorporated in the diacetylene or polydiacetylene colloids are preferably in an amount of about 4-10 molar percent. Attaching the diacetylene colloids via the thiol/epoxide reaction has been shown to give better materials for some assays as compared to attaching the colloids via other reactions.

Some of the advantages of attaching diacetylene and polydiacetylene colloids to surfaces include: (a) attachment makes changing the solutions around the colloids much quicker and easier (simply decant off the old solution and add new) than the dialysis or column techniques needed for free colloids; (b) similarly, attachment allows easy addition and removal of reagents that react with the colloids to add or alter functionality; (c) attachment allows use of salts, pH conditions and reagents that would cause free colloids to aggregate and drop out of solution; (d) attachment allows patterning of the colloids on surfaces, and (e) attachment still leaves the colloids open to the surrounding solution on most sides, unlike the sol-gel encapsulation method which sequesters the colloids from the bulk solution, so it seems likely, though the inventors are not bound thereby, that the kinetics of reaction, binding etc are not slowed as much.

Diacetylene and polydiacetylene colloids can be attached to glass fiber membranes, or other membrane types, in commercially available 96-well (or 384 well or other multi-well formats) plates; they can also be attached to glass fiber membranes, glass, or other materials that are incorporated in 96 well (or 384 well or other multi-well formats) plates, or transferred to 96 well (or 384 well or other multi-well formats) plates. The advantages of attaching the colloids to porous membranes are that the user can direct analyte solution past the sensing materials and thus improve contact by filtering the analyte solution through the membrane (Dai J. et al. Anal. Chem. 2006, 78(1), 135-140) and can also concentrate analytes that are larger than the membrane pore size at the membrane surface where the sensing materials are located.

The diacetylene or polydiacetylene colloids may contain components that are substrates or ligands for specific targets. Further chemistry can be performed on the diacetylene or polydiacetylene colloids after they are attached to the glass to add functionality, such as addition of antibodies, other proteins, peptides, phages, viruses, nucleic acids, aptamers, DNA and/or RNA sequences, calixarenes, crown ethers, cyclodextrans, sugars etc.

The advantages of using the polydiacetylene emission (or emission of fluorophores incorporated in the polydiacetylene 3-D tethered arrays) for detection of analytes are both greater sensitivity over absorbance methods and the ability to use opaque supports (including filter membranes, porous glass etc) that cannot be used in colorimetric detection. The polydiacetylene colloids may be in a relatively low emissive state at the beginning of the assay and become more fluorescent; or they may be already fluorescent and become either less fluorescent or more fluorescent in response to interaction with the analyte.

According to another aspect of the present disclosure a supported three-dimensional array of a polydiacetylene backbone bonded to and supported by a solid support is provided. The solid support has a layer of polyelectrolyte on the surface that is to be in contact with the three-dimensional array. The array optionally has incorporated therein a substrate with direct affinity for an analyte or can function as a binder to an analyte or can react with an analyte.

According to this aspect of the present disclosure, the solid support is not limited to, but can employ, if desired, the support having thiol groups or at least one member selected from the group consisting of epoxy groups and maleimide groups thereon. The solid support optionally may have a reactive monomer at the surface. Examples of reactive monomers include but are not limited to styrenes, acrylates, methacrylates, norbornenes, cyclooctadienes, acetylenes, diacetylenes etc. The reactive monomer may be used to grow or graft the polyelectrolyte upon the surface.

The polyelectrolytes, include polylysine, poly(glutamic acid), poly(acrylic acid), poly(allylamine), proteins, self assembled monolayers (SAMS), hydrophilic polymers, hydrophobic polymers, polyvinylpyrrolidinone, polyethylene glycol (PEG), bovine serum albumin (BSA), peptides, polynucleic acids, nucleic acids, salts, metals, arrays of small molecules, and combinations thereof. Layers of different polyelectrolytes may be applied to the surface, including alternating layers with different charges. The colloids may covalently bound to the functional groups of the polyelectrolyte at the surface or interact through noncovalent interactions such as ionic, dipole and hydrogen bonding, including hybridization of nucleic acid sequences. For example, mixed cellulose ester membranes can be coated with polylysine or polylysine and BSA; polymerized polydiacetylene liposomes can then be filtered onto the coated membrane to form supported liposomes. The polydiacetylene liposomes may contain ligands or substrates for analytes, or may be functionalized after being attached to the coated membrane; they are then used for fluorescent detection of analytes.

The poly(lysine) polyelectrolytes appropriate for the materials and methods of this disclosure are preferably the hydrobromide salts, with molecular weights in the range 15,000-150,000 g/mol, and more preferable with molecular weights in the range 70,000-150,000 g/mol.

A still further aspect of this disclosure is concerned with a supported three-dimensional array of a polydiacetylene backbone bonded to and supported by a solid support. A multiple atom linker is located between the three-dimensional array of the polydiacetylene backbone and the solid support. The array optionally has incorporated therein a substrate that has direct affinity for an analyte or can function as a binder to an analyte or can react with an analyte.

According to this aspect of the present disclosure, the solid support is not limited to the support having thiol groups or at least one member selected from the group consisting of epoxy groups and maleimide groups thereon.

Examples of suitable multiple atom linkers include, but are not limited to, hydrocarbon chains (typically 1-22 carbon atoms), cyclohexyl groups, aromatic groups (typically hydrocarbon or hetero rings containing 5-12 carbons and more typically phenyl), oligio(ethylene glycol)s and combinations thereof. Examples of suitable terminal functional groups for the multiple atom linkers include, but are not limited to, carboxylic acids, aldehydes, esters, amines, thiols, maleimides, epoxides, alcohols, alkoxides, halides, ammoniums, azides, mesylates, tosylates, hydrazides, and any other functional group that can be displaced or substituted. Some specific multiple atom linkers appropriate for the materials and methods of this disclosure include, but are not limited to, amino-dPEG₄™ acid, thiol-dPEG₄™ acid, amino-dPEG₈™ acid, and thiol-dPEG₈™ acid.

The presence of the linker is advantageous since it moves the detecting reaction away from possible surface effects of the solid support.

If desired, two or all three of the above techniques of attachment can be used together.

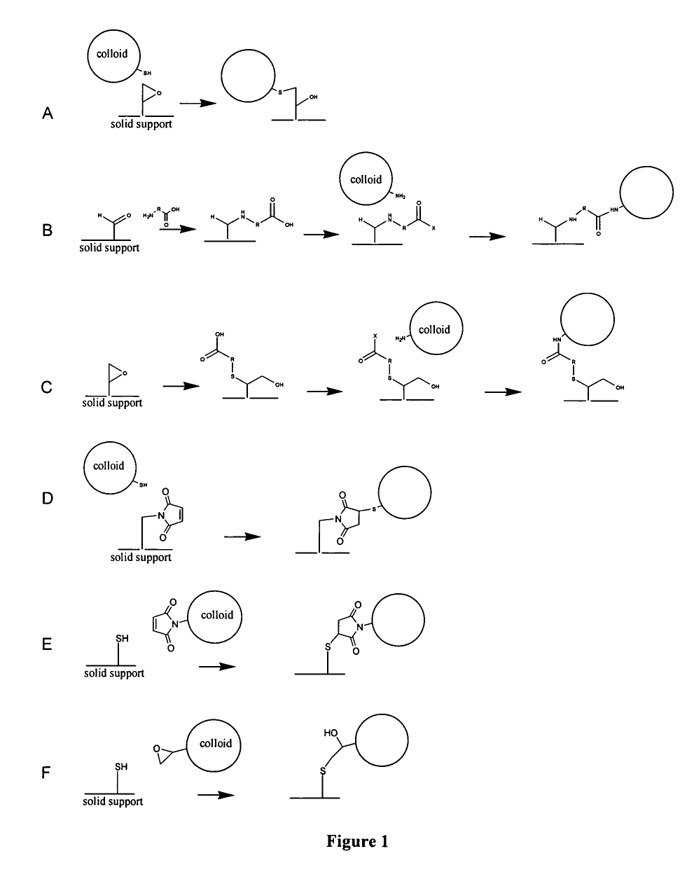

Examples of methods of attaching diacetylene and polydiacetylene colloids to glass or other solid supports that can be used with the last two embodiments include, but are not limited to:
1) glass or other solid support with aldehyde groups and diacetylene or polydiacetylene colloids with amines where the aldehyde and amine first reversibly form a Schiff base. The Schiff base is then irreversibly reduced to an amine;
2) glass or other solid support with epoxide groups and diacetylene or polydiacetylene colloids with thiols and/or amines and/or hydroxide groups that can react with the epoxides in a ring-opening reaction (FIG. 1A);
3) glass or other solid support with aldehyde groups that are reacted with α-amino-ω-carboxy hydrocarbon or PEG linkers—the amine group reacts with the aldehyde via reductive amination and the carboxy group is then converted to an active ester which reacts with amine groups in diacetylene or polydiacetylene colloids forming an amide bond (FIG. 1B);
4) glass or other solid support with epoxide groups that react with α-thiol-co-carboxy hydrocarbon or PEG linkers—the thiol opens the epoxide forming a thio-ether bond and the carboxy group is then converted to an active ester which reacts with amine groups in diacetylene or polydiacetylene colloids forming an amide bond (FIG. 1C);
5) glass or other solid support with maleimide groups and diacetylene or polydiacetylene colloids with thiols that can add to the maleimides to form a chemical bond (FIG. 1D);
6) glass or other solid support with thiol groups and diacetylene or polydiacetylene colloids with maleimide groups that can react with the thiols to form a chemical bond (FIG. 1E);
7) glass or other solid support with thiol groups and diacetylene or polydiacetylene colloids with epoxy groups that can react with the thiols to form a chemical bond (FIG. 1F)

Further solid supports include, but are not limited to, plastics such as polystyrene, fluorocarbon polymers such as polytetrafluorethylene (PTFE), polyolefins such as polypropylene, polyvinylidenedifluoride (PVDF), polycarbonate and poly(meth)acrylate; metals such as gold, silver, platinum, silicon etc; metal oxides; ceramics; and other (non-silica) glasses, etc. Particular membrane supports include mixed cellulose ester membranes, polyester membranes, nylon membranes, polycarbonate membranes, hydrophilic PVDF membranes, hydrophilic PTFE membranes, and hydrophilic polypropylene membranes, etc.

The polydiacetylene colloids attached to membranes, or other surfaces, may incorporate non-diacetylene lipids and be exposed to small molecules; the change in the polydiacetylene emission is then correlated to the small molecules' druglike properties (lipophilicity, membrane permeation, tissue uptake, volume of distribution, blood brain barrier penetration etc). Alternatively, the change in emission of polydiacetylene colloids attached to membrane surfaces upon exposure to solution of small molecules may be used to detect the presence and with suitable calibration, the concentration of the small molecules. The polydiacetylene colloids may be at the start of the assay in either a non-fluorescent or fluorescent state; the emission may either rise or fall in response to interaction with the small molecules.

As discussed above, supported three-dimensional arrays of this disclosure can be used for detecting of an analyte in a sample by measuring a change in fluorescence and/or phosphorescence. The analyte/substrate pairs include receptor proteins as either an analyte or substrate. When the receptor protein is an analyte it may be stabilized in a lipid array, such as a lipid raft, micelle, vesicle etc. Receptor proteins include, but are not limited to, G-coupled protein receptors, opioid receptors, acetylcholine receptors, adrenergic receptors, neurotensin receptors, serotonin receptors, opsin receptors, motilin receptors, ion channels, p-glycoprotein, etc.

Also, supported three-dimensional arrays of this disclosure can be used for evaluating at least one of the ionization state of a compound, the organic/water partition coefficient and lipophilicity, oral absorption, the volume of distribution of a compound, the distribution of a compound into different tissues, the ability of a compound to diffuse into cell membranes and the partitioning of a compound into cell organelles, by measuring the effect on the array by detecting the change in fluorescence or phosphorescence. Supported three-dimensional arrays of this disclosure can be used for evaluating the binding of a compound to a protein.

Examples of some analyte and substrate systems that can be used in the present invention are as follows:
The analyte is an enzyme and the substrate is a reactive substrate of that enzyme.
The analyte is the reactive substrate of the enzyme and the substrate is the enzyme.
The analyte binds to a receptor protein and the substrate is the receptor protein.
The analyte is a receptor protein and the substrate can bind to the receptor protein.
The analyte is an antigen and the substrate is the antibody of that antigen.
The analyte is an antigen and the substrate is a fragment of the antibody of that antigen.
The analyte is an antibody or antibody fragment and the substrate is the antigen of that antibody.
The analyte is an antibody or antibody fragment and the substrate is the epitope of that antibody.
The analyte is a microorganism and fragment thereof and the substrate is a phage particle.
The analyte is a microorganism and fragment thereof and the substrate is a nucleic acid aptamer.
The analyte is a microorganism and fragment thereof and the substrate is a peptide.
The analyte is a microorganism and fragment thereof and the substrate is a peptide.
The analyte is a protein and the substrate is a phage particle.
The analyte is a protein and the substrate is a nucleic acid aptamer.
The analyte is a protein and the substrate is a peptide.
The analyte is a nucleic acid sequence and the substrate contains a complimentary nucleic acid sequence capable of hybridization with the analyte.

The following non-limiting examples are presented to further facilitate an understanding of the present disclosure:

In the following examples, unless otherwise stated, the diacetylene fatty acids are purchased from GFS or synthesized in-house. Acetylene compounds are purchased from GFS or Lancaster. Trimethoxy and triethoxy silanes are purchased from United Chemical Technologies. Reagents are obtained from Sigma Aldrich, Fisher Scientific, Pierce and Quanta Biodesigns. Organic fluorophores are obtained from Molecular Probes. Fluorophores and include: 5-(((4-(4,4-difluoro-5-(2-thienyl)-4-bora-3a,4a-diaza-s-indacene-3-yl)phenoxy)acetyl)amino)pentylamine hydrochloride (BODIPY® TR cadaverine) (1) and 1,1′-dioctadecyl-3,3,3′,3′-tetramethylindodicarbocyanine, 4-chlorobenzenesulfonate salt (DIC-18(5)) (2), 1,2-Dimyristoyl-sn-glycero-3-phosphocholine (DMPC), 1,2-Dioleoyl-sn-glycero-3-phosphocholine (DOPC), 1,2-Dioleoyl-sn-glycero-3-phosphoethanolamine (DOPE) and 1,2-dipalmitoyl-sn-glycero-3-phosphothioethanol, sodium salt (DPPT) are purchased from Avanti Polar Lipids. Bee venom phospholipase A₂ (PLA₂) is purchased from Sigma-Aldrich and 12-epi-scalaradial from CalBiochem. Ricin toxin is purchased from Vector Laboratories. Anti ricin polyclonal antibodies, anti Bacillus globigii (BG) polyclonal antibodies, and BG spores are obtained from the U.S. Department of Defense JPED-CBD Critical Reagents Program. Additional BG spores are generated from Bacillus atrophaeus var. niger, obtained from the ATCC (48337), grown and sporulated according to literature methods (Schaeffer P et al. Proc. Natl. Acad. Sci. 1965, 704-711). Cryptosporidium parvum oocysts are obtained from Waterborne, and anti C. parvum polyclonal antibodies are obtained from the NIH AIDS Reagent Program (originally donated by Dr. Joseph Crabb). The antibodies are purified by size exclusion chromatography in 0.1M sodium phosphate/0.1M sodium chloride at pH 7.0, and stored frozen at 4.35 mg/mL. Water is deionized to a final resistance of >17.5 MΩ and sterilized with UV light (Aurora).

Diacetylene fatty acids that are not commercially available are synthesized according to literature methods by the copper (1) mediated coupling of the appropriate 1-iodo-1-yne hydrocarbon and ω-yne carboxylic acid (Tieke B. et al, Angew. Chem. Int. Ed. Eng., 1976, 15(12), 764-765). N-(2-Hydroxyethyl)-amide derivatives (Spevak et al, J. Am. Chem. Soc. 1993, 115, 1146-1147), and mono phosphate derivatives (Hub H—H.; Hupfer B.; Koch H.; Ringsdorf H., Angew. Chem. Int. Ed. Engl. 1980, 19(11), 938-940) are synthesized by literature methods. 1-amino-10,12-pentacosadiyne (P—NH₂) is synthesized in four steps from 10,12-pentacosadiynoic acid: the acid in anhydrous tetrahydrofuran at 0° C. is reduced to the alcohol through treatment with lithium aluminum hydride (2.5 equivalents) in ether for two hours, the alcohol is then converted to the mesylate by treatment with mesyl chloride (6 equivalents) in methylene chloride with diisopropyl ethyl amine over 30 minutes, the mesylate is displaced by sodium azide (1.5 equivalents) in dimethyl formamide at 70° C. over one hour and the azide in tetrahydrofuran is reduced to the amine with lithium aluminum hydride (2 equivalents) in ether at 0° C. over one hour. The amine is treated with aqueous hydrochloride to convert it to the hydrochloride salt for storage.

Anti-BG phage were developed in-house according to the following methods. A Ph.D-0.7 Phage Display Peptide Library Kit containing a random displayed 7-mer library fused to the M13 phage, host E. coli K₁₂ ER2738, and appropriate sequencing primers were purchased from New England Biolabs. The 7-mer phage display peptide library (complexity 2.8×10⁹ sequences) was screened according to a literature procedure (Knurr J. et al, Appl. Environ. Microbio., 2003, 69(11), 6841-6847) using gravity centrifugation to collect spore-phage complexes. Double washed B. globigii spores (2×10⁹) were mixed with the library phage (2×10¹¹) in 1 mL of sterile filtered TBST (50 mM Tris, 150 mM NaCl, 0.5% Tween-20, pH 7.5) and allowed to bind for 10 minutes at room temperature. Spore-phage complexes were collected by gravity centrifugation (12,000×g) for 10 minutes at 4° C. The supernatant was decanted and the complexes were washed 10× with ice-cold TBST. A sample from the fifth wash was saved in order to assess the amount of non-specific phage virions that were washed from the complexes. Specific phage virions were eluted from the spore-phage complexes with the addition of 1 ml elution buffer (0.2 mM glycine-HCl, 1 mg/ml BSA, pH 2.2) and then gently rocked for 5 minutes at room temperature. A final centrifugation for 5 minutes isolated the eluted phage, which was retained in the supernatant. This eluted phage was transferred to a fresh microcentrifuge tube and 150 μl of 1 M Tris-HCl pH 9.1 was added to neutralize the phage. The eluted phage was amplified according to manufacturer's protocol and a titer of each stock was determined. The first round's amplified phage was used as the input phage in round two. The entire procedure was repeated for three rounds total of biopanning. Twenty clones from each method were selected for sequencing and analysis. Genomic ssDNA was extracted and purified also according to the manufacturer's protocol and templates sent for DNA sequence analysis (GeneWhiz).

Probe sonication is achieved with Biologics 300 V/T Ultrasonic homogenizer fitted with an intermediate tip. Photopolymerization is achieved using a UV-oven capable of delivering calibrated energy doses of UV light around 254 nm. Assay data are collected using a Molecular Devices Spectramax Gemini EM plate reader and a Wallach Victor²V plate reader. ¹H and ¹³C spectra are obtained by Acorn NMR, Livermore Calif.

Silica glass fiber filters are obtained from Millipore Corporation, both as disks and in 96-well plates with TiO₂/barex housing. The filters come in B (700μ thick; 1.0μ effective pores) and C (240 g thick; 1.2 g effective pores) forms that are primarily distinguished by their different thicknesses. Individual filter circles are punched from the larger filters.

Diacetylene colloid solutions are prepared according to general methods presented in the literature (Hupfer B. et al, Chem. Phys. Lipids, 1983, 33, 355-374; Spevak et al, J. Am. Chem. Soc., 1993, 115, 1146-supplementary materials; Reichart A. et al. J. Am. Chem. Soc., 1995, 117, 829-supplementary materials) by drying organic solutions of the diacetylene surfactants together with any lipophilic additives, adding water or buffer to bring the combined materials to 1 mM or 2 mM overall, probe sonication to disperse the materials and filtration through a 0.8 μm pore size cellulose acetate filter.

### EXAMPLE 1

Epoxy and aldehyde groups are added to the surfaces of the glass filter fibers as follows: the filter circles or filter plates are washed with absolute ethanol (200 μL/well in plates; enough to cover for free filter circles). A 4% (vol/vol) solution of 3-glycidoxy propyl trimethoxy silane (for epoxy groups) or 3-propylaldehyde triethoxy silane (for aldehyde groups) in 95% EtOH/5% 0.1M acetate buffer at pH 4.8 is shaken for 2-3 minutes. Washed filters are covered with this solution; the solution is added to filters in plates at 100 μL/well. The filters and silane solutions are shaken for 20 minutes, the silane solutions decanted and the filters washed 1-2 times with absolute ethanol. The filters are then cured at room temperature in a desiccator charged with anhydrous calcium sulfate, for a minimum of 30 hours.

### EXAMPLE 2

A diacetylene colloid solution is prepared consisting of 60% 6,8-docosadiynoic acid/33% DMPC/7% DPPT and incorporating fluorophore 1 at a ratio of one 1:200 lipids, at 2 mM lipid overall, in a combination of 4.8 mL of argon-sparged H₂O and 0.2 mL 0.1M sodium borate, pH 7.9. After cooling to room temperature 100 μL/well of the colloid solutions are added to a Millipore glass filter B plates, which were previously functionalized with 3-glycidoxy propyl trimethoxy silane, as described in Example 1. The plate remains standing for 3 hours at room temperature and then the colloid solutions are decanted. The wells are washed with 200 μL/well of 10 mM sodium borate at pH 8.0 (borate buffer), two times. The wells are charged with 90 μL of borate buffer and 10 μL of 10% B-mercaptoethanol in borate buffer. The plate remains at room temperature for approximately one hour, and the solutions are decanted. The wells are then washed with 200 μL/well of borate buffer once, then with PBS (10 mM NaPO₄/138 mM NaCV2.7 mM KCl at pH 7.4) two times. 150 μL of 50 mM TRIS at pH 7.8 is added per well and the plate stored at 4° C. overnight. The plate is then exposed to 0.4 J/cm² of UV light at 254 nm. The plate is stored at 4° C. until use.

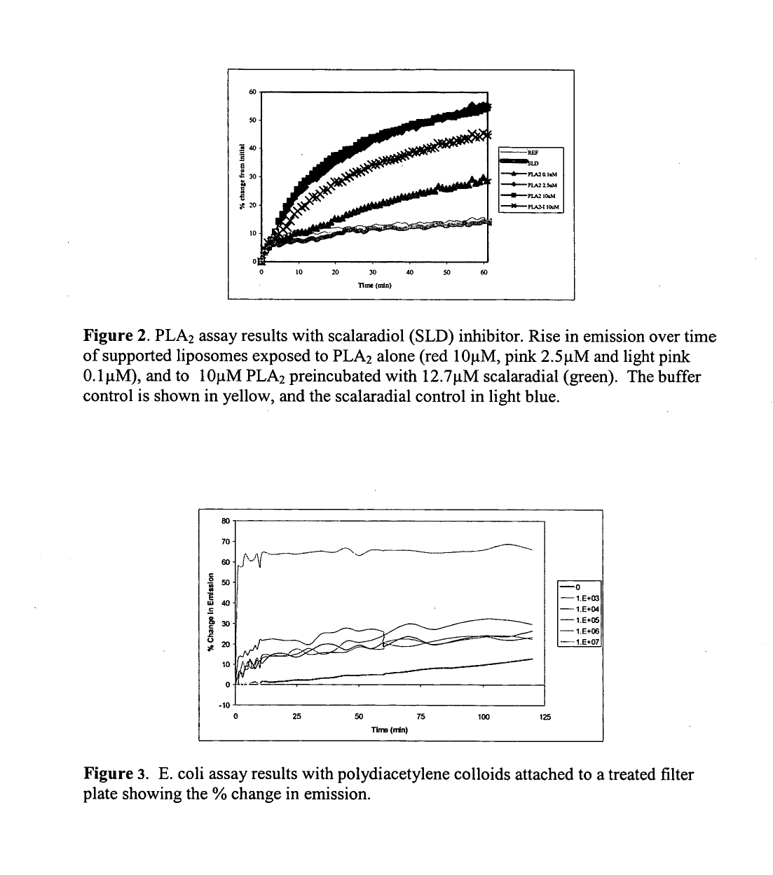

An assay to detect the activity of PLA₂ and inhibition of that activity by 12-epi-scalaradial is performed. The plate is decanted and 90 μL/well of 50 mM TRIS pH 7.8 is added; the initial emission of the plate is read in the Gemini plate reader at room temperature (λₑₓ=470 nm; λₑₘ=560 nm and 640 nm with cutoff filters at 530 nm and 610 nm respectively). PLA₂ is dissolved in 10 mM TRIS/150 mM NaCl at pH 8.9; scalaradial is dissolved at 11.4 mM in methanol. Solutions are prepared with PLA₂ at 100 μM, 25 μM and 1 μM, with 100 μM PLA₂ combined with 127 μM scalaradial, and with 127 μM scalaradial alone. 10 mM TRIS/150 mM NaCl, pH 8.9 is used as a control. Appropriate amounts of methanol are added to solutions without scalaradial so that the concentration of methanol (1.1%) is the same in all samples. The solutions are incubated with shaking for 1 hour, and then 10 μL of each are added to the plate wells, in quadruplicate. The plate is shaken and the emission read in the Gemini plate reader (same excitation and emission setup as for initial read) over 99 minutes. The percentage changes in emission of quadruplicate wells are calculated; the initial emission measurements have been made before the addition of the enzyme and inhibitor solutions. The results clearly show detection of PLA₂ activity at 10, 2.5 and 0.1 μM in the assay, as well as partial inhibition of the 10 μM PLA₂ by the schalaradiol. See FIG. 2 which shows the rise in emission at 640 nm of supported liposomes exposed to PLA₂ alone (red 10 μM, pink 2.5 μM and light pink 0.1 μM), and to 10 μM PLA₂ with 12.7 μM scalaradial (green). The buffer control is shown in yellow, and the scalaradial control in light blue.

### EXAMPLE 3

A diacetylene colloid solution is prepared consisting of 60% 6,8-docosadiynoic acid/33% DMPC/7% P—NH₂ and incorporating fluorophore 1 at a ratio of one 1:200 lipids, at 2 mM lipid overall, in a combination of 4.8 mL of argon-sparged H₂O and 0.2 mL 0.1M sodium borate, pH 9.5. After cooling to room temperature, 100 μL/well of the colloid solutions are added to a Millipore glass filter B plates, which were previously functionalized with 3-propylcarboxyaldehyde triethoxy silane, as described in Example 1. The plate remains for 100 minutes at room temperature. Sodium cyanoborohydride is dissolved at 5M in 1M sodium hydroxide and then diluted to 0.5M with 0.1M sodium borate pH 8.5. 15 μL of this solution is added to the wells, the plate remains for 70 minutes and then 10 μL/well of 1M diethanolamine in H₂O is added. The plate remains for 1 hour and then is decanted. The wells are washed two times with PBS (10 mM NaPO₄/138 mM NaCl/2.7 mM KCl at pH 7.4) two times. 150 μL of 50 mM TRIS at pH 7.8 is added per well and the plate stored at 4° C. overnight. The plate is then exposed to 0.4 J/cm² of UV light at 254 nm. The plate is stored at 4° C. until use.

An assay to detect the activity of PLA₂ and inhibition of that activity by 12-epi-scalaradial is performed. The plate is decanted and 90 μL/well of 50 mM TRIS pH 7.8 is added; the initial emission of the plate is read in the Gemini plate reader at room temperature λₑₓ=470 nm; λₑₘ=560 nm and 640 nm with cutoff filters at 530 nm and 610 nm respectively). PLA₂ is dissolved in 10 mM TRIS/150 mM NaCl at pH 8.9; scalaradial is dissolved at 11.4 mM in methanol. Solutions are prepared with PLA₂ at 100 μM, 25 μM and 1 μM, with 100 μM PLA₂ combined with 127 μM scalaradial, and with 127 μM scalaradial alone. 10 mM TRIS/150 mM NaCl, pH 8.9 is used as a control. Appropriate amounts of methanol are added to solutions without scalaradial so that the concentration of methanol (1.1%) is the same in all samples. The solutions are incubated with shaking for 1 hour, and then 10 μL of each are added to the plate wells, in quadruplicate. The plate is shaken and the emission is read in the Gemini plate reader (same excitation and emission setup as for initial read) over 99 minutes. The percentage changes in emission of quadruplicate wells are calculated; the initial emission measurements being made before the addition of the enzyme and inhibitor solutions. Unlike in Example 2, there is no detection of PLA₂ activity or scalaradial inhibition. These two examples taken together suggest that attaching the diacetylene colloids via the thiol/epoxide reaction gives better materials for this assay than attaching the colloids via reductive amination.

### EXAMPLE 4

A diacetylene colloid solution consisting of 70% 6,8-pentacosadiynoic acid/20% 10,12-pentacosadiyn-1-amine/10% DPPT and incorporating fluorophore 2, at one 2:300 lipids, is prepared at 2 mM lipid overall in 4.5 mL argon-sparged H₂O and 0.5 mL 0.1M sodium borate, pH 7.9. 100 μL/well of this solution is added to Millipore C filter circles in a white polystyrene 96 well plate (Corning); the filters were previously functionalized with 3-glycidoxy propyl trimethoxy silane, as described in Example 1. The plate remains at room temperature for approximately 5 hours; viton o-rings are added to the wells to hold the membranes down around the 4.5 hour point. At 5 hours the colloid solutions are dumped and the wells washed one time with 10 mM sodium borate, pH 8.0. The wells are then treated with 100 μL/well of 0.1M B-mercaptoethanol in 10 mM sodium borate, pH 8.0, for one hour with gentle shaking. The plate is decanted and the wells washed with 200 uL/well of 10 mM sodium borate (twice) and PBS (twice), with vigorous shaking during each wash. 200 μL/well of PBS is added to the plate and the plate stored at 4° C. overnight.

The following day the plate is decanted and the wells charged with 90 μL PBS and 10 μL 3 mM bis(sulfosuccinimidyl)suberate (BS³, Pierce) in PBS. The plate is shaken gently for one hour, decanted and the wells washed with 200 μL/well of PBS three times, with vigorous shaking. The wells are charged with 90 μL PBS and 10 μL of anti-BG polyclonal antibodies, diluted to 1 mg/mL with PBS. The plate is shaken slowly for over four hours, decanted and washed three times with PBS as above. The wells are then washed once with 100 mM CaCl₂ in 10 mM MOPS, pH 7.4 (Ca-M), and charged with 200 μL of Ca-M. The plate is stored at 4° C. overnight. The following day the plate is decanted and exposed to 0.3 J/cm² of UV light at 254 nm. The glass filter membranes are then transferred with their o-rings to a black polystyrene plate containing 50 μL/well of Ca-M. The excess liquid is decanted and 100 μL/well of Ca-M is added.

The emission of the plate is read in the Gemini plate reader with λₑₓ=470 nm; λₑₘ=560 nm, 640 nm and 675 nm (550 nm, 610 nm and 665 nm cutoff filters respectively). The plate is decanted, the wells charged with 90 μL Ca-M and 10 μL of BG spore dilutions in water are added to the wells in quadruplicates. The plate is shaken in the reader and the emission read over approximately 5 hours; analyses at 90 and 95% confidence levels of the averaged change in emission over the first hour shows detection of the BG spores over the control at 2×10⁵, 2×10⁴, and 2×10³ spores per well.

### EXAMPLE 5

A diacetylene colloid solution consisting of 70% 6,8-pentacosadiynoic acid/20% 10,12-pentacosadiyn-1-amine/10% DPPT and incorporating fluorophore 2, at one 2:300 lipids, is prepared at 2 mM lipid overall in 4.5 mL argon-sparged H₂O and 0.5 mL 0.1M sodium borate, pH 7.9. 100 μL/well of this solution is added to Millipore C filters in a white polystyrene 96 well plate (Corning); the filters were previously functionalized with 3-glycidoxy propyl trimethoxy silane, as described in Example 1. The plate remains at room temperature for approximately 5 hours; viton o-rings are added to the wells to hold the membranes down around the 4.5 hour point. At 5 hours the colloid solutions are dumped and the wells washed one time with 10 mM sodium borate, pH 8.0. The wells are then treated with 100 μL/well of 0.1M B-mercaptoethanol in 10 mM sodium borate, pH 8.0, for one hour with gentle shaking. The plate is decanted and the wells washed with 200 uL/well of 10 mM sodium borate (twice) and PBS (twice), with vigorous shaking during each wash. 200 μL/well of PBS is added to the plate and the plate stored at 4° C. overnight.

The following day the plate is decanted and the wells charged with 90 μL PBS and 10 μL 3 mM bis(sulfosuccinimidyl)suberate (BS³, Pierce) in PBS. The plate is shaken gently for one hour, decanted and the wells washed with 200 μL/well of PBS three times, with vigorous shaking. The wells are charged with 90 μL PBS and 4×10¹¹ M13 bacteriophage, biopanned against BG spores three times, displaying the peptide sequence APAPLLL.

The plate is shaken for 4.5 h at moderate speed then decanted and the wells washed with PBS two times and 50 mM CaCl₂/10 mM MOPS pH 7.4 (Ca-M) two times. The plate is stored overnight with 200 μL/well of Ca-M at 4° C. The following day the plate is decanted and exposed to 0.3 J/cm² of UV light at 254 nm. The glass filter membranes are then transferred with their O-rings to a black polystyrene plate containing 10 μL/well of Ca-M. The plate is decanted and 100 μL/well of Ca-M added.

The emission of the plate is read in the Gemini plate reader with λₑₓ=470 nm; λₑₘ=560 nm, 640 nm and 675 nm (550 nm, 610 nm and 665 nm cutoff filters respectively). The plate is decanted, the wells charged with 90 μL Ca-M and 10 μL of BG spore and Cryptosprodium parvum oocyst dilutions in water are added to the wells in quadruplicates. The plate is shaken in the reader and the emission read over 5.25 h; analyses at 90 and 95% confidence levels of the averaged change in emission from the initial readings shows detection of the BG spores over the control at 1×10⁵, 1×10⁴, and 1×10³ spores, preferentially over the C. parvum oocysts.

### EXAMPLE 6

Diacetylene colloids are prepared at 1 mM with the formulations: 40% N-(2-Hydroxyethyl)-10,12-pentacosadiynamide (PCDA-EtOH)/24% DOPC/10% DOPE/4% DPPT/22% cholesterol, and 76% PCDA-EtOH/10% trimyristin/10% tripalmitin/4% DPPT, in 4.75 mL argon-sparged H₂O with 0.25 mL 0.1M sodium borate pH 7.9. The colloids are polymerized with 0.2 J/cm² of UV light at 254 nm, and heated at 72° C. for 5 m under argon. The solutions are added at 100 μL/well to Millipore C filters in a white polystyrene 96 well plate (Corning); the filters were previously functionalized with 3-glycidoxy propyl trimethoxy silane, as described in Example 1, and the plate incubated at room temperature for 4 h, for 16 h at 4° C., and 1 h at room temperature. Viton O-rings are added to the plate, the plate decanted and washed with 5 mM HEPES buffer pH 6.8, four times. The membranes and O-rings are transferred to a black polystyrene plate with rinsing in 5 mM HEPES pH 6.8 en route. The membranes are then stored in 5 mM HEPES pH 6.8 100 μL/well, at 4° C.

The emission of the plate (now at room temperature) is read with a Victor²-V plate reader with λₑₓ=485 nm; λₑₘ=560 nm, 642 nm. The plate is decanted and the membranes are exposed to 100 μL/well of 100 μM solutions of drug and drug-like compounds in 5 mM HEPES pH 6.8 and 2% DMSO and the emissions monitored. The changes in the emission versus the initial emission are calculated and compared to the lipophilicity and adipose tissue uptake of the compounds.

### EXAMPLE 7

A solution of diacetylene colloids is prepared from 95% 10,12-pentacosadiynoic acid/5% DPPT with one 1:200 lipids incorporated in 5 mM HEPES pH 7.4. 100 μL/well of this solution is added to Millipore C filters in a white polystyrene 96 well plate (Corning); the filters were previously functionalized with 3-glycidoxy propyl trimethoxy silane, as described in Example 1. The plate is incubated at room temperature for 4.5 h then put at 4° C. for 21 h. Viton o-rings are added to the wells and the plate decanted. The plate is washed with 5 mM HEPES pH 7.4 two times and chilled at 4° C. with 200 μL/well of 5 mM HEPES pH 7.4 buffer. The plate is then decanted and the colloids polymerized with 0.2 j/cm² of UV light around 254 nm. The plate is stored at 4° C. with 100 μL/well of 5 mM HEPES pH 7.4.

The emission of the plate (now at room temperature) is read with a Victor²-V plate reader with λₑₓ=485 nm; λₑₘ=560 nm, 642 nm. The plate is decanted and the membranes are exposed to 100 μL/well of 100 μM solutions of drug and drug-like compounds in 5 mM HEPES pH 7.4 and 2% DMSO and the emissions monitored. The changes in the emission versus the initial emission are calculated and compared to a reference (DMSO), to determine detection of the compounds by the attached liposomes.

### EXAMPLE 8

A 2 mM solution of thiol-dPEG₄ (Quanta Biodesign) in 0.1M sodium borate at pH 7.91 is diluted 1:1 with H₂O. 100 μL/well is then added to Millipore B filters in a plate; the filters were previously treated with 3-glycidoxy propyl trimethoxy silane, as described in Example 1. The plate is then covered and shaken for 250 minutes. The solution is decanted and the wells washed three times (200 μL/well) with 10 mM sodium phosphate at pH 6.5 (Buffer A) with decanting between washes. The final wash is decanted and the plate stored at 4° C. overnight.

The following day, a diacetylene colloid solution is prepared from 80% 6,8-docosadiynoic acid and 20% dipalmitoyl-sn-glycerophosphatidyl ethanolamine with fluorophore 1 incorporated at one 1:400 lipids, at 2 mM lipid in one part 0.1M sodium borate pH 9.52 to 25 parts H₂O.

While the colloid solution is prepared, sulfo-N-hydroxysuccinimide (sulfo-NHS, Sigma-Aldich) was dissolved at 22.8 mg/mL Buffer A and 1-ethyl-(3-dimethylaminopropyl-3-ethylcarbodiimide hydrochloride) (EDC, Sigma-Aldrich) is dissolved at 23.5 mg/mL Buffer A. 120.6 μL of the sulfo-NHS solution and 145.4 μL of the EDC solution are combined and 25 mL Buffer A added. This solution is added to the plate at 200 μL/well, and the plate shaken for 105 minutes. The plate is then washed with Buffer A three times with 200 μL/well per wash.

100 μL/well of the colloid solution was added to the plate and the plate shaken slowly, covered, for two hours. The colloid solution is then decanted and the wells washed three times with Buffer A. The wells are then charged with 100 μL/well PBS (10 mM sodium phosphate/138 mM sodium chloride/2.7 mM potassium chloride at pH 7.4) and the plate stored at 4° C. overnight.

The following day the plate is decanted and the wells charged with 90 μL PBS and 10 μL 1.6 mM bis(sulfosuccinimidyl)suberate (BS3, Pierce) in PBS. The plate is shaken slowly for 45 minutes, then the solution decanted and the wells washed with 200 μL/well PBS three times. The wells are then charged with 90 μL PBS and 10 μL of anti-C. parvum oocyst polyclonal antibodies diluted to 1 mg/mL with PBS. The plate is shaken slowly for three hours, decanted and washed three times with PBS (200 μL/well). The wells are then charged with 100 μL/well PBS (10 mM sodium phosphate/138 mM sodium chloride/2.7 mM potassium chloride at pH 7.4) and the plate stored at 4° C. overnight. The following day the PBS is decanted from the plate and the plate exposed to 0.2 J/cm² of UV light at 254 nm to polymerized the diacetylene colloids.

The plate wells are charged with 100 μL PBS and the emission is read in the Gemini plate reader with λₑₓ=470 nm; λₑₘ=560 nm and 640 nm (550 nm and 610 nm cutoff filters respectively). The PBS is then decanted. The wells are then charged with 90 μL/well of PBS and 10 μL/well of C. parvum oocysts diluted in H₂O added to give triplicate samples. The emission of the plate is then monitored over two hours with 3 s shakes before each reading. The % change from initial emission are calculated for each well and then averaged. The attached polydiacetylene colloids show a greater rise in emission when exposed to 10³ and 10⁴ oocysts/well than when exposed to the control (H₂O)—outside the standard deviations of the averages. The attached colloids are more responsive to the lower number of oocysts.

### EXAMPLE 9

A 96-well mixed cellulose ester filter plate (pore size 0.45μ) from Millipore is charged with 100 μL/well of a 0.1 mg/ml poly-D-lysine solution in water. The plate is incubated at room temperature for three hours and the solution is decanted. The plate is air dried overnight. Each well is then filled with 275 μL of a 1% bovine serum albumin solution prepared in PBS pH 7.4. The plate is incubated at room temperature for three hours and then decanted and air dried overnight.

### EXAMPLE 10

A diacetylene colloid solution is prepared from PCDA-PO₄ with fluorophore 1 incorporated, at one 1:200 lipids. Hinge reduction of a Biodesign rabbit anti-E. coli polyclonal antibody was accomplished by incubating 4 mg of antibody with 6 mg/mL of MEA in 50 mL volume of 0.1 M Na₂HPO₄, 0.15 M NaCl and 10 mM EDTA buffer at pH 7.2. This reaction was incubated at 37° C. for 90 min. Dialysis against 5 mM NaOAc/50 mM NaCl/50 mM NaCl/10 mM EDTA at pH 7.0 removed unreacted MEA. Hinge-reduced E. coli antibodies were covalently linked to a lipid tail purchased from Northern Lipids, N-(6-Maleimidohexanoyl)-1,2-distearoyl-sn-glycero-3-phosphoethanolamine (MXDSPE) through the maleimide group. A 20-fold molar excess of the lipid tail is added to the dialyzed anti-E. coli and incubated at 4° C. overnight. The antibody-MXDSPE reaction is dialyzed three times against PBS pH 7.4 to remove unbound MXDSPE. HPLC using a size exclusion column confirmed the relative purity of the antibody at each stage of the labeling and purification process. Based upon A₂₈₀ measurements, 80% of the original amount of antibody is retained. The antibodies with the attached lipid tails are incorporated into liposomes through detergent dialysis (Huang A. et al, J. Biol. Chem., 1980, 255(17), 8015-8018): 10 mLs of the diacetylene colloid solution were combined with 520 μL/mL of a 12.5% deoxycholate solution, 1 mL of 10 mM NaPO₄/15 mM KPO₄/1.37 M NaCl/27 mM KCl at pH 6.5, and 100 μL of MXDSPE. This solution is dialyzed against 9 L of PBS pH 7.4 at 4° C. three times using 10,000 MWCO membranes, with one dialysis running overnight.

100 μL of the diacetylene colloid-antibody conjugate solution is added to each well of the filter plate, previously treated as in Example 6, and the plate irradiated with 0.2 J/cm² of UV light at 254 nm. The fluorescence of the polymerized colloids is measured using 470 nm excitation and 560 and 640 nm emission wavelengths. The plate is filtered at 350 Torr. E. coli cells are counted and diluted to various concentrations in a 50 mM CsCl solution. 100 uL of the various E. coli dilutions are added to wells in quadruplicate, bringing the final assay concentration of CsCl to 25 mM. The change in fluorescence is measured over time and compared to the initial signal (before the samples were added). Changes from the initial fluorescence signal of the attached liposomes could be observed at 560 nm over a period of two hours following addition of cells; there is a clear rise above the control for samples with 1×10⁷ to 1×10³/mL E. coli (FIG. 3).

### EXAMPLE 11

A diacetylene colloid solution is prepared from 86% 10,12-pentacosadiynoic acid/10% 1-Palmitoyl-2-oleoyl-sn-glycero-3-phosphocholine (POPC)/4% ganglioside GM1 with fluorophore 1 incorporated at one 1:200 lipids. After sonication, the formulation is stored overnight at 10° C. and then at 4° C. for nine days prior to use. The formulation is then chilled on ice and irradiated with 0.06 J/cm² of UV light at 254 nm.

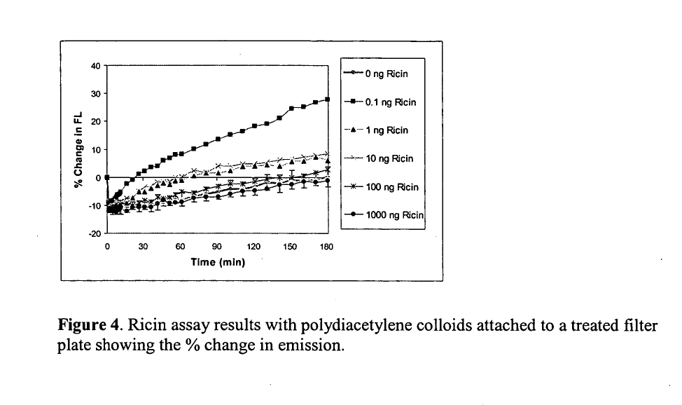

100 μL/well of the polymerized colloid solution is then added to a filter plate, pretreated as described in Example 6. The plate is chilled for thirty minutes at 4° C. and then filtered over 30 minutes at 350 Torr. The plate is stored overnight at 4° C. under humid conditions. Dilutions of ricin are added in 10 mM NaPO₄/150 mM NaCl/0.08% NaN₃ and the emission of the plate monitored. The 0.1 ng ricin sample rises significantly above the control; there is an inverse dose response with the highest ricin amount (1 μg) showing no response different from control (FIG. 4).

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such or other embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference.

1. A supported three-dimensional array of a polydiacetylene backbone bonded to and supported by a solid support having at least one member selected from the group consisting of thiol group and amine group or at least one member selected from the group consisting of epoxy group and maleimide group, and wherein the polydiacetylene backbone contains the other of thiol group and/or amine group or the epoxy group and/or maleimide group for reacting with the corresponding thiol groups or epoxy groups and/or maleimide groups on the support; provided that when an amine is used the other group is an epoxy; and wherein said array optionally has incorporated therein a substrate that has direct affinity for an analyte or can function as a binder to an analyte or can react with an analyte.

2. A method for the detection of an analyte in a sample, which comprises contacting the sample to be tested to the supported three-dimensional array according to claim 1 wherein said array has incorporated therein a substrate that has direct affinity for an analyte or can function as a binder to an analyte or can react with al analyte;
and detecting the change in fluorescence or phosphorescence to indicate the presence of the analyte.

3. A method for evaluating at least one of the ionization state of a compound, the volume of distribution of a compound, the distribution of a compound into different tissues, the organic/water partition coefficient and lipophilicity of a compound, the oral absorption of a compound, the ability of a compound to diffuse into cell supports and the partitioning of a compound into cell organelles, which comprises exposing the supported three-dimensional array according to claim 1 to the compound to be evaluated; and
measuring the effect on the array by detecting the change in fluorescence or phosphorescence of the array.

4. A method for evaluating the binding of a compound to a protein, which comprises exposing the supported three-dimensional array according to claim 1 to a solution of the compound to be evaluated post or during exposure to the protein;
measuring the effect on the array of the compound to be evaluated by detecting the change in fluorescence or phosphorescence,
comparing the change to changes in fluorescence or phosphorescence from exposure of the array of the kind being used to a reference compound in solution.

5. A method for screening a plurality of samples containing different species, which comprises exposing an attached three-dimensional array of a polydiacetylene backbone according to claim 1 to the samples to be evaluated; wherein the array is capable of hetero-detection:
detecting the change in fluorescence or phosphorescence of the array, and
comparing the change to a previously determined change in fluorescence or phosphorescence of the array to determine whether the species are present in the samples. In a further refinement, comparison with calibration curves allows determination of the concentration of the species.

6. A supported three-dimensional array of a polydiacetylene backbone bonded to and supported by a solid support having a layer of polyelectrolyte thereon and wherein said array optionally has incorporated therein a substrate that has direct affinity for an analyte or can function as a binder to an analyte or can react with an analyte.

7. A method for the detection of an analyte in a sample, which comprises contacting the sample to be tested to the supported tree-dimensional array according to claim 6 and wherein said array has incorporated therein a substrate that has direct affinity for an analyte or can function as a binder to an analyte or can react with an analyte;
and detecting the change in fluorescence or phosphorescence to indicate the presence of the analyte.

8. A method for evaluating at least one of the ionization state of a compound, the organic/water partition coefficient and lipophilicity of a compound, the oral absorption of a compound, the volume of distribution of a compound, the distribution of a compound into different tissues, the ability of a compound to diffuse into cell membranes and the partitioning of a compound into cell organelles, which comprises exposing the supported free-dimensional array according to claim 6 to the compound to be evaluated; and
measuring the effect on the array by detecting the change in fluorescence or phosphorescence of the array.

9. A method for evaluating the binding of a compound to a protein, which comprises exposing the supported three-dimensional array according to claim 6 to a solution of the compound to be evaluated post or during exposure to the protein;
measuring the effect on the array of the compound to be evaluated by detecting the change in fluorescence or phosphorescence,
comparing the change to changes in fluorescence or phosphorescence from exposure of the array of the kind being used to a reference compound in solution.

10. A method for screening a plurality of samples containing different species, which comprises exposing an attached three-dimensional array of a polydiacetylene backbone according to claim 6 to the samples to be evaluated; wherein the array is capable of hetero-detection:
detecting the change in fluorescence or phosphorescence of the array, and
comparing the change to a previously determined change in fluorescence or phosphorescence of the array to determine whether the species are present in the samples. In a further refinement, comparison with calibration curves allows determination of the concentration of the species.

11. A supported three-dimensional array of a polydiacetylene backbone bonded to and supported by a solid support which comprises a multiple atom linker between the three dimensional array of a polydiacetylene backbone and the solid support and wherein the array optionally has incorporated therein a substrate that direct affinity for an analyte or can function as a binder to an analyte or can react with an analyte.

12. A method for the detection of an analyte in a sample, which comprises contacting the sample to be tested to the supported three-dimensional array according to claim 11 and wherein said array has incorporated therein a substrate that has direct affinity for an analyte or can function as a binder to an analyte or can react with an analyte;
and detecting the change in fluorescence to indicate the presence of the analyte.

13. A method for evaluating at least one of the ionization state of a compound, the volume of distribution of a compound, the distribution of a compound into different tissues, the organic/water partition coefficient and lipophilicity of a compound, oral absorption of a compound, the ability of a compound to diffuse into cell membranes and the partitioning of a compound into cell organelles, which comprises exposing the supported three-dimensional array according to claim 11 to the compound to be evaluated; and
measuring the effect on the array by detecting the change in fluorescence or phosphorescence.

14. A method for evaluating the binding of a compound to a protein, which comprises exposing the supported three-dimensional array according to claim 11 to a solution of the compound to be evaluated post or during exposure to the protein;
measuring the effect on the array of the compound to be evaluated by detecting the change in fluorescence or phosphorescence,
comparing the change to changes in fluorescence or phosphorescence from exposure of the array of the kind being used to a reference compound in solution.

15. A method for screening a plurality of samples containing different species, which comprises exposing an attached three-dimensional array of a polydiacetylene backbone according to claim 11 to the samples to be evaluated; wherein the array is capable of hetero-detection:
detecting the change in fluorescence or phosphorescence of the array, and
comparing the change to a previously determined change in fluorescence or phosphorescence of the array to determine whether the species are present in the samples. In a further refinement, comparison with calibration curves allows determination of the concentration of the species.

16-27. (canceled)

28. The method of claim 2, wherein the polydiacetylene of the array is in the non-fluorescent form, exhibiting a fluorescent signal that is about 1-3 times that of the background and less than that of the corresponding fluorescent form.

29. The method of claim 2, wherein the substrate includes a ligand.

30. The method of claim 2, wherein the substrate includes a reactive substrate.

31. The method of claim 2, wherein the three-dimensional array further comprises a fluorophore and wherein the change in fluorescence of the polydiacetylene array is monitored.

32. The method of claim 2, wherein the three-dimensional array further comprises a fluorophore and wherein the change in fluorescence of the fluorophore is monitored.

33. The supported three-dimensional array of claim 1 wherein the thiol is at least one member selected from the group consisting of 1-dodecanethiol, 1-undecanethiol, 1-decane thiol, and 1,2-dipalmitoyl-sn-glyceohosphatidylethanethiol.

34. The supported three-dimensional array of claim 1 wherein the three-dimensional array of a polydiacetylene backbone further comprises an amine

35. The supported three-dimensional array of claim 35 wherein the three-dimensional array of a polydiacetylene backbone is attached via addition of said amine to aldehyde groups on the support forming a Schiff base.

36. The supported three-dimensional array of claim 1 wherein the attachment of said three-dimensional array of a polydiacetylene backbone further includes a Schiff base, which has been reduced to an amine.

37. The supported three-dimensional array of claim 1, wherein the thiol is reacted with one end of a multiple atom linker attached to the solid support, forming a thio ether bond.

38. The supported three-dimensional array of claim 34, wherein the amine is reacted with one end of a multiple atom linker attached to the solid support, forming an amide bond.

39. The supported three-dimensional array of claim 1, wherein the solid support is a planar glass surface.

40. (canceled)

41. The supported three-dimensional array claim 1, wherein the solid support is a glass fiber membrane.

42. The supported three-dimensional array of claim 1, wherein the solid support is porous glass.

43. (canceled)

44. (canceled)

45. (canceled)

46. (canceled)

47. The supported three-dimensional array of claim 1 wherein said epoxy group is at least one member selected from the group consisting of 3-glycidoxy propyl trimethoxy silane, 3-glycidoxypropyl triethoxy silane, 3-glycidoxypropylmethyl dimethoxy silane, 3-glycidoxypropylmethyl diethoxy silane, epoxycyclohexylethyltiimethoxysilane, other mono, di and trialkoxy silanes that contain epoxy groups, and mono, di and trihalo silanes that incorporate epoxy groups.

48. The supported three-dimensional array of claim 11 wherein said multiple atom linkers are at least one member selected from the group consisting of hydrocarbon chains, cyclohexyl groups, aromatic groups, oligio(ethylene glycol)s and combinations thereof.

49. The supported three-dimensional array of claim 42 wherein said multiple atom linkers further contain terminal functional groups selected for the group of carboxylic acids, aldehydes, esters, amines, thiols, maleimides, epoxides, alcohols, alkoxides, halides, ammoniums, azides, mesylates, tosylates, hydrazides, and combinations thereof.

50. The supported three-dimensional array of claim 1 wherein said support is a glass support having epoxy groups thereon and said array contains said thiol groups.

51. The supported three-dimensional array of claim 6 wherein the polyelectrolyte comprises polylysine or polylysine with a layer of bovine serum albumin above the polylysine.

52. (canceled)

53. (canceled)

54. (canceled)
